(19) United States
(12) Patent Application Publication
(10) Pub. No.: US 2026/0315070 A1
(43) Pub. Date: Oct. 15, 2026

(54) Wound Closure Patch

(71) Applicant: Curaform LLC, Greeley, CO (US)

(72) Inventor: Frank Dwayne Morgan, Windsor, CO (US)

(73) Assignee: Curaform LLC, Greeley, CO (US)

(21) Appl. No.: 19/440,074

(22) Filed: Jan. 5, 2026

Related U.S. Application Data
(60) Provisional application No. 63/787,022, filed on Apr. 11, 2025.
(60) Provisional application No. 63/802,738, filed on May 9, 2025.

(51) Int. Cl. A61B 17/08; A61B 17/00; A61F 13/00; A61F 13/02; A61K 31/12; A61K 33/38; A61K 35/64; A61K 36/886; A61L 15/22; A61L 15/44

(52) U.S. Cl. CPC A61B 17/085; A61F 13/00063; A61F 13/0266; A61F 13/0289; A61K 31/12; A61K 33/38; A61K 35/64; A61K 36/886; A61L 15/225; A61L 15/44; A61B 2017/00893; A61B 2017/081

(57) ABSTRACT

A wound closure patch comprising an outer contractible layer, the contractible layer being a stimuli-responsive polymer, an intermediate layer, and an adhesive layer, wherein a stimulus is presented to the contractible layer thereby contracting the outer contractible layer and bringing edges of a wound into close proximity. The wound closure patch may further comprise a pad inserted into a central channel of the wound closure patch fitted over the wound. The wound closure patch may further comprise a peelable layer on the adhesive layer and/or adhesive backed wings fitted to the outer contractible layer, the wings having optionally a peelable layer fitted to the adhesive backing. A method of forming and method of using the wound closure patch of the present disclosure is presented.

The present disclosure is an application for patent under 35 U.S.C. 111(a). The present application claims the benefit of U.S. Provisional application No. 63/787,022, filed Apr. 11, 2025, and U.S. Provisional application No. 63/802,738, filed May 9, 2025, each incorporated by reference herein.

### I. FIELD OF THE INVENTION

The present disclosure is related to wound treatment devices and methods of making wound treatment devices and methods of using wound treatment devices. Specifically, a wound care patch for bringing and maintaining wound edges in close proximity to speed healing.

### II. BACKGROUND OF THE INVENTION

Traditional wound closure techniques such as sutures, staples, and tissue adhesives often involve discomfort, require technical expertise, or result in cosmetically undesirable outcomes. There is a need for a non-invasive, easy-to-apply, sterile wound closure system that minimizes pain, reduces infection risk, and improves cosmetic results, especially in pediatric, field, and outpatient care settings.

### III. SUMMARY OF THE INVENTION

The disclosure provides a wound closure system comprising an adhesive patch with a contractible polymer layer. The patch includes an adhesive backing layer for securing the patch to skin, a thermal insulating intermediate layer, and an activated contractible polymer film on the upper surface. The central portion of the patch may be trimmed to expose the wound site directly.

After manually approximating the wound edges, a technician or user applies the patch to clean, dry, pre-treated skin surrounding the wound. A targeted heat source, such as an IR wand or low-power laser, may be applied to the shrinkable polymer surface, causing controlled contraction of the film. This contraction generates mild, uniform inward tension that draws the wound edges together and holds them in place. The patch remains adhered for several days, acting as a protective dressing and facilitating wound healing without sutures or staples.

In other terms, the present disclosure provides a wound support system comprising a customizable adhesive patch with a heat-shrinkable polymer top layer, an insulating middle layer, and a skin-safe adhesive bottom layer. The unique architecture includes two spaced lateral adhesive zones that form a central channel over the wound. This channel allows for the insertion and removal of a dressing element, such as a TELFA® strip, beneath the shrinkable layer.

Following manual approximation of the wound edges, the user applies the patch such that the two adhesive rails straddle the wound. Applying gentle heat, the contractible or shrinkable layer contracts, generating mild, uniform tension across the wound channel to maintain edge approximation. The device also allows an internal gauze or absorbent strip to be replaced without removing the patch, preserving wound stability while supporting hygiene and topical treatment.

As such, the disclosure comprises a wound closure patch comprising: an outer contractible layer extending to a contractible layer periphery; an intermediate layer joined to said outer contractible layer, said intermediate layer including a pair of segments disposed in spaced apart relation defining a central channel having opposite channel ends open at said contractible layer periphery; and an adhesive layer joined to said intermediate layer. The wound closure patch further comprises an aperture open between opposed surfaces of said outer contractible layer, said aperture disposed in said outer contractible layer over said central channel. The wound closure patch further comprises wherein said outer contractible layer comprises a stimuli-responsive polymer which contracts when exposed to an activation stimulus. This wound closure patch further comprises wherein said activation stimulus comprises one or more of a temperature stimulus, a light stimulus, a chemical stimulus, an electric stimulus, a magnetic field stimulus, and a humidity stimulus. This wound closure patch further comprises wherein said stimuli-responsive polymer comprises one or more of a PET, polylactic acid, a polyurethane, and an ethylene-vinyl acetate.

The wound closure patch further comprises wherein said outer contractible layer comprises a thermal shrink polymer which contracts in response to a thermal stimulus. This wound closure patch further comprises wherein said thermal stimulus comprises one or more of thermal conduction, thermal convection, and thermal radiation. This wound closure patch further comprises wherein said thermal stimulus heats said thermal shrink polymer to a temperature of about 30 °C (about 86 °F) to about 80 °C (about 176 °F). This wound closure patch further comprises wherein said temperature is selected from the group consisting of: about 30 °C to about 40 °C, about 35 °C to about 45 °C, about 40 °C to about 50 °C, about 45 °C to about 55 °C, about 50 °C to about 60 °C, about 55 °C to about 65 °C, about 60 °C to about 70 °C, and about 65 °C to about 75 °C.

The wound closure patch further comprises wherein said outer contractible layer has a thickness of about 0.20 mm to about 1.0 mm. This wound closure patch further comprises wherein said outer contractible layer has a thickness selected from the group consisting of about 0.25 mm to about 0.35 mm, about 0.30 mm to about 0.40 mm, about 0.35 mm to about 0.45 mm, about 0.40 mm to about 0.50 mm, about 0.45 mm to about 0.55 mm, about 0.50 mm to about 0.60 mm, about 0.55 mm to about 0.65 mm, about 0.60 mm to about 0.70 mm, about 0.65 mm to about 0.75 mm, about 0.70 mm to about 0.80 mm, about 0.75 mm to about 0.85 mm, about 0.80 mm to about 0.90 mm, and about 0.85 to about 0.95 mm.

The wound closure patch further comprises wherein said outer contractible layer comprises one or more of a PET, polyurethane film, a polyolefin film, a polyester film, a polynorbornene film, a polyvinyl acetate film, an epoxy-thermoset polymer film, a polymethyl methacrylate film, a thiol-ene film, ethylene-vinyl acetate (EVA), shape-memory polymers, and combinations thereof. This wound closure patch further comprises wherein said polyurethane film layer is selected from the group consisting of a thermoplastic polyurethane film, a polyester urethane film, a polyether urethane film, a polycarbonate urethane film, and combinations thereof. This wound closure patch further comprises wherein said polyolefin film is selected from the group consisting of: a polyethylene film, an ethylene-vinyl acetate film, a polyethylene-co-butylene film, and combinations thereof.

The wound closure patch further comprises wherein said intermediate layer joined to said outer contractible layer comprises a thermal insulator. This wound closure patch further comprises wherein said thermal insulator comprises one or more of an open cell foam, a closed cell foam, a hydrogel, and combinations thereof. This wound closure patch further comprises wherein said intermediate layer joined to said outer contractible film layer comprises one or more of: a polyurethane foam, a reticulated polyurethane foam, a hydrocellular foam, a silicone foam, a hydrofiber foam, alginate foam, a chitosan foam, a collagen foam, a hydrogel foam, a polyvinylpyrrolidone foam, a polyethylene glycol foam, a polylactic-co-glycolic acid foam, a polylactic acid foam, a polybutylene succinate foam, a polyhydroxyalkanoates foam, a poly-ε-caprolactone foam, a polyethylene foam, a cross-linked polyethylene foam, a polypropylene foam, a polyethylene-vinyl acetate foam, a polyvinyl chloride foam, a polyvinyl formal foam, a polyvinyl alcohol foam, a polyimide foam, a polystyrene foam, a melamine foam, a nitrile rubber foam, a hydroxyapatite-based foams, and combinations thereof.

The wound closure patch further comprises wherein said intermediate layer has a thickness of about 1.00 mm to about 5.00 mm. This wound closure patch further comprises wherein said intermediate layer has a thickness selected from the group consisting of: about 1.50 mm to about 2.50 mm, about 2.00 mm to about 3.00 mm, about 2.50 mm to about 3.50 mm, about 3.0 mm to about 4.0 mm, and about 3.5 mm to about 4.50 mm.

The wound closure patch further comprises wherein said adhesive layer comprises a biocompatible adhesive layer. This wound closure patch further comprises wherein said biocompatible adhesive layer comprises one or more of: a cyanoacrylate adhesive, a 2-octyl-cyanoacrylate adhesive, a n-butyl cyanoacrylate adhesive, a silicone adhesive, an acrylic adhesive, a UV-curable acrylate adhesive, a polyurethane adhesive, a hydrogel adhesive, a collagen adhesive, a chitosan adhesive, an albumin adhesive, a dextran adhesive, a gelatin adhesive, a poly ethylene glycol adhesive, a polyacrylic acid adhesive, a polylactic-co-glycolic adhesive, and combinations thereof.

The wound closure patch further comprises a pad disposed in said central channel. This wound closure patch further comprises wherein said pad comprises a wound dressing. This wound closure patch further comprises wherein said wound dressing comprises one or more of: a gauze dressing, a transparent film dressing, a hydrocolloid dressing, and combinations thereof. The wound closure patch further comprises a tether connected to said pad, said tether configured to pass through said central channel, said pad drawn by said tether into said central channel. This wound closure patch further comprises a medicament disposed on said pad. This wound closure patch further comprises wherein said medicament comprises one or more of: an antibiotic, an antiseptic, an ointment, silver, honey, aloe vera, curcumin, and nonsteroidal anti-inflammatory.

The wound closure patch further comprises a peelable layer joined to said adhesive layer, said peelable layer peelable from said adhesive layer. The wound closure patch further comprises adhesive backed wings attached to the contractible layer periphery and flanking each of the opposite channel ends. This wound closure patch further comprises wherein the adhesive backed wings comprise a peelable layer attached to the adhesive. The wound closure patch further comprises wherein the outer contractible layer comprises a transparent portion covering at least a portion of the central channel.

Further, a method is provided for making a wound closure patch of the present disclosure, wherein the method comprises: forming an outer contractible layer; adhering at least one intermediate layer section to the outer contractible layer; forming an adhesive layer on the at least one intermediate layer section. The method may further comprise wherein the outer contractible layer comprises a shape stimuli-responsive polymer. The method may further comprise wherein said activation stimulus comprises one or more of a heat stimulus, a light stimulus, a chemical stimulus, an electric stimulus, an electric field stimulus, a magnetic field stimulus, and a humidity stimulus. The method may further comprise wherein the at least one intermediate layer section comprises a thermal insulator. The method further comprises applying a peelable layer to the adhesive layer. The method may further comprise wherein at least two intermediate layer sections are adhered to the outer contractible layer at a first end and a second end of the outer contractible layer forming a central channel with open ends disposed at a contractible layer periphery. This method may further comprise forming adhesive backed wings on the contractible layer periphery spanning at least the open ends of the central channel. This method may further comprise applying a peelable layer on the adhesive of the adhesive backed wings.

The disclosure further comprises a method of treating a wound with exposed edges, the method comprising: cleaning the wound and surrounding skin; drying the surrounding skin; adhering the wound treatment patch over the wound such that the central channel covers the wound; and activating the activation stimulus to pull the exposed edges of the wound into close contact. The method may further comprise fitting a pad into the central channel, wherein the pad comprises one or more of a gauze dressing, a transparent film dressing, and a hydrocolloid dressing.

In other embodiments, the disclosure comprises a wound support device comprising a heat-shrinkable polymer top layer, two spaced adhesive side layers, and a central channel for insertion of a removable absorbent dressing. The wound support device may further comprise wherein the top layer contracts upon application of heat, creating gentle inward pressure to support wound edge approximation. The wound support device may further comprise wherein the dressing element is a TELFA® strip, antimicrobial pad, or honey-infused gauze, or wherein the dressing element may be removed and replaced without disturbing the adhesive attachment to the skin, or wherein the polymer top layer is transparent and forms a tensioned canopy over the wound channel. The wound support device may further comprise a thermal indicator ink, antimicrobial coatings, or breathable adhesive material.

In another embodiment, a method for supporting wound healing comprises: approximating wound edges, applying a dual-rail adhesive patch with a shrinkable polymer film, inserting an absorbent strip beneath the film, and activating the film with heat to maintain gentle support, wherein the absorbent strip can be removed and replaced during the healing process. The disclosure further comprises device or method of the disclosed wherein the heat is applied by a chemical heat pack or infrared wand.

In other embodiments, the wound closure device comprises a shrinkable polymer top layer, an insulating intermediate layer, and an adhesive bottom layer, wherein the shrinkable layer contracts upon exposure to heat, thereby applying inward tension to approximate wound edges. The device may further comprise wherein the central portion is trimmable to expose the wound. The device may further comprise wherein the shrinkable polymer is selected from PET, polyolefin, or a shape-memory polymer. The device may further comprise wherein the insulating layer is comprised of foam, gel, or other thermally resistant material. The device may further comprise wherein the shrinkable polymer layer is semi-transparent or transparent.

The disclosure further comprises a method for closing a wound comprising: approximating the wound edges, applying a trimmable adhesive patch over the wound, and directing heat to the polymer layer to shrink the patch and apply tension to the wound margins.

Naturally, further objects of the embodiments are disclosed throughout other areas of the specification, drawings, and claims.

### IV. BRIEF DESCRIPTION OF THE DRAWINGS

FIG. 1 is an illustration of the wound closure patch of the present disclosure in use on a subject.

FIG. 2 is a schematic drawing of a side view of one embodiment of the wound closure patch of the present disclosure.

FIG. 3 is a schematic drawing of a side view of one embodiment of the wound closure patch of the present disclosure.

FIG. 4 is a schematic drawing of a top view of one embodiment of the wound closure patch of the present disclosure.

FIG. 5 is a schematic drawing of a bottom view of one embodiment of the wound closure patch of the present disclosure.

FIG. 6 is a schematic drawing of a bottom view of one embodiment of the wound closure patch of the present disclosure.

FIG. 7 is a schematic drawing of a bottom view of one embodiment of the wound closure patch of the present disclosure.

FIG. 8 is a schematic drawing of a bottom view of one embodiment of the wound closure patch of the present disclosure.

FIG. 9 is a top perspective view of one embodiment of the wound closure patch of the present disclosure.

FIG. 10 is a bottom perspective view of one embodiment of the wound closure patch of the present disclosure.

FIG. 11 is a top view of one embodiment of the wound closure patch of the present disclosure.

FIG. 12 is a bottom view of one embodiment of the wound closure patch of the present disclosure.

FIG. 13 is a right side view of one embodiment of the wound closure patch of the present disclosure.

FIG. 14 is a left side view of one embodiment of the wound closure patch of the present disclosure.

FIG. 15 is a front view of one embodiment of the wound closure patch of the present disclosure.

FIG. 16 is a back view of one embodiment of the wound closure patch of the present disclosure.

FIGS. 17-19 are schematic illustrations of embodiments of a pad of the wound closure patch of the present disclosure.

FIG. 20 is a schematic illustration of a method of making the wound closure patch of the present disclosure.

FIG. 21 is a schematic illustration of a method of using the wound closure patch of the present disclosure.

### V. DETAILED DESCRIPTION OF THE INVENTION

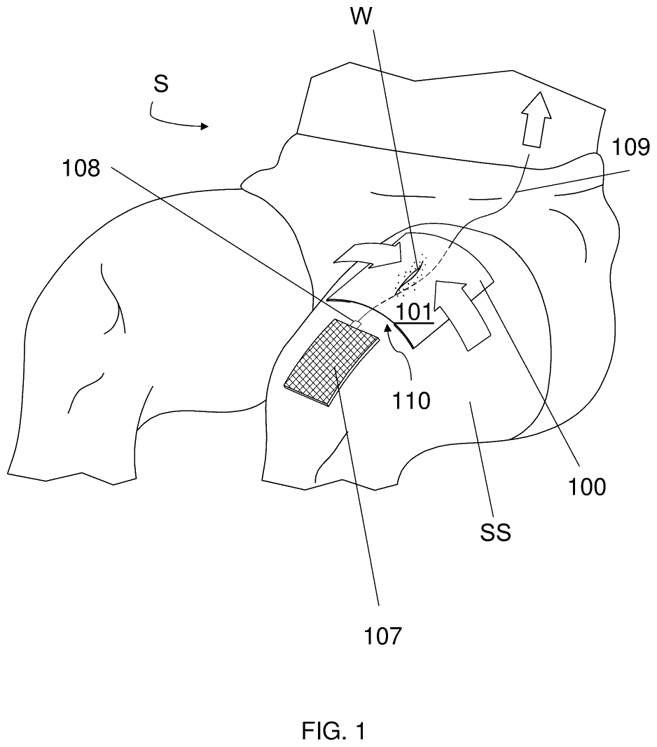

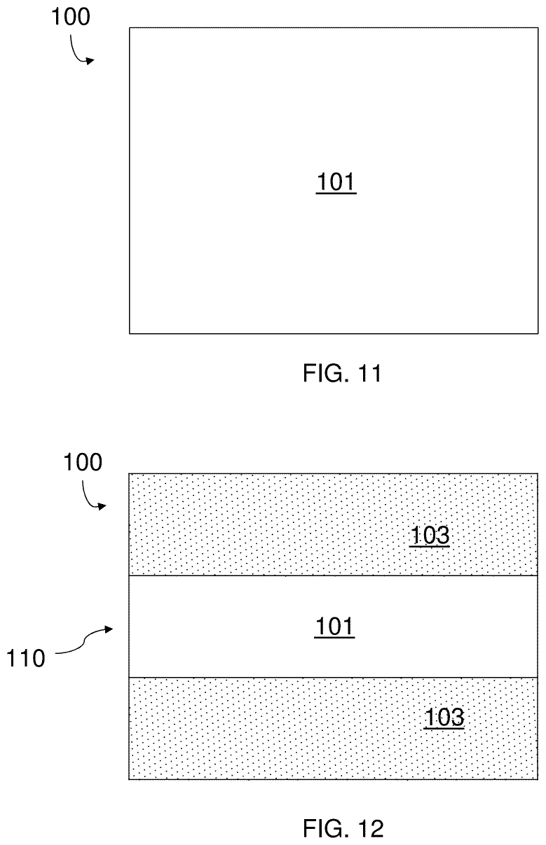

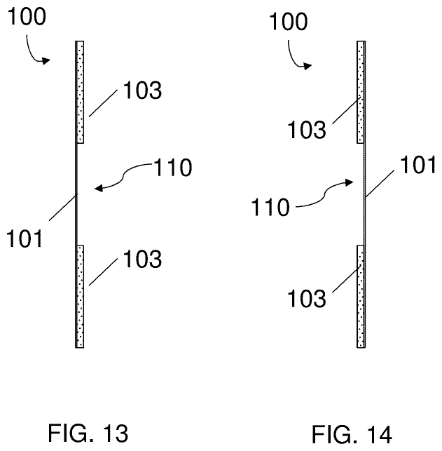

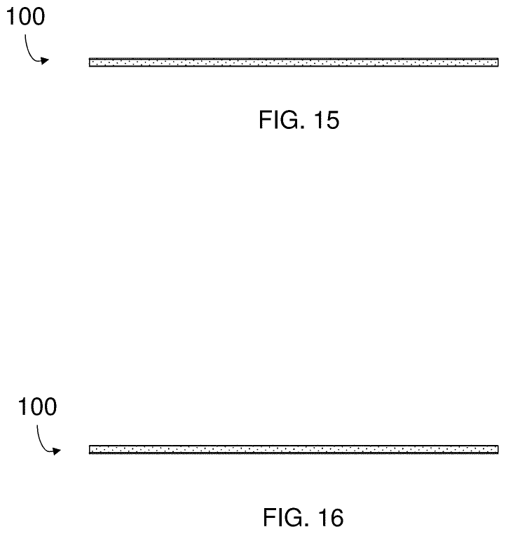

Generally, referring to FIGS. 1 through 21, the present disclosure is directed to a wound closure patch and method for making and using embodiments of the wound closure patch described and shown herein. The wound closure patch comprises at least three joined layers being an outer contractible layer, an intermediate layer having at least one intermediate layer segment, and an adhesive layer joined to the intermediate layer. For purposes of the following disclosure, the following definitions are presented.

### Definitions

As used herein, a “contractible layer” comprises a layer of flexible material capable of contracting in response to an added stimuli, examples of which may include but are not limited to a stimuli-responsive polymer.

As used herein, a “stimuli-responsive polymer” or “shrinkable polymer film” or “shape-memory polymer” comprises a polymer material that changes physical or chemical properties in response to an external trigger. For purposes of the present application, the stimuli-responsive polymer contracts when exposed to the external trigger or activation stimulus.

As used herein, an “activation stimulus” comprises an external trigger that imparts physical and/or chemical changes on a stimuli-responsive polymer causing it to contract, wherein the activation stimulus may include but is not limited to temperature, pH, light, magnetic or electric fields, electricity, chemicals, elements, compounds, molecules, or biomolecules.

As used herein, a “thermal shrink polymer” refers to a type of polymer that shrinks when exposed to a heat or heat activation stimulus.

As used herein, an “open cell foam” comprises a material with interconnected openings or cells that allow moisture and/or air to pass through it.

As used herein, a “closed cell foam” comprises a material comprising non-interconnected openings or cells that may be moisture resistant and provide an airtight seal.

As used herein, a “hydrogel” comprises a three-dimensional (3D) network of polymer chains that may absorb moisture or water, and may comprise a natural hydrogel such as collagen, gelatin, or hyaluronic acid. A hydrogel may be biocompatible and may comprise a synthetic hydrogel such as but not limited to a synthetic polymer like polyethylene glycol (PEG) or polyvinyl alcohol (PVA).

As used herein, a “hydrocolloid” comprises a type of colloid being a particle or molecule that forms a gel with it comes into contact with moisture and wherein a colloid is defined as a homogeneous non-crystalline substance consisting of molecules or particles such that when dispersed through a second substance including but not limited to gels, sols, and emulsions the particles do not settle in the suspension.

As used herein, an “adhesive” comprises any substance that binds objects together via surface attachment, examples of which may include but are not limited to glue, cement, paste, and mucilage with examples such as PVA, epoxy, and pressure-sensitive adhesives.

As used herein, a “biocompatible adhesive” or “biocompatible adhesive layer” or “bio-adhesive” comprises an adhesive that is a material safe for use on or in the human body, examples of which may include but are not limited to acrylics, silicones, and hydrocolloids.

As used herein, a “medicament” refers to a substance used for medical treatment.

As used herein, a “nonsteroidal anti-inflammatory” refers to a class of medications used to relieve pain, reduce fever, and decrease inflammation. They work by blocking the production of enzymes called cyclooxygenase (COX) which are responsible for creating prostaglandins that cause inflammation, pain, and fever. Common examples include ibuprofen, e.g. ADVIL® or MOTRIN®, and naproxen (ALEVE®), which are available over-the-counter, as well as prescription NSAIDs like celecoxib (CELEBREX®).

As used herein, a “subject” may comprise a living organism, in examples a human or an animal, with a wound in need of treatment thereof.

As used herein, a “medical aid” may be a person or support tool, whether a traditional tool like tweezers or other tool designed for placing bandages or dressings, or robotic tool or machine, or artificial intelligence machine, that places the wound closure patch of the present disclosure or aids in placing the wound closure patch of the present disclosure onto or over a wound of a subject. The medical aid may be a trained medical technician, nurse, doctor, nurse practitioner, physician's assistant, or other medical professional.

### Description

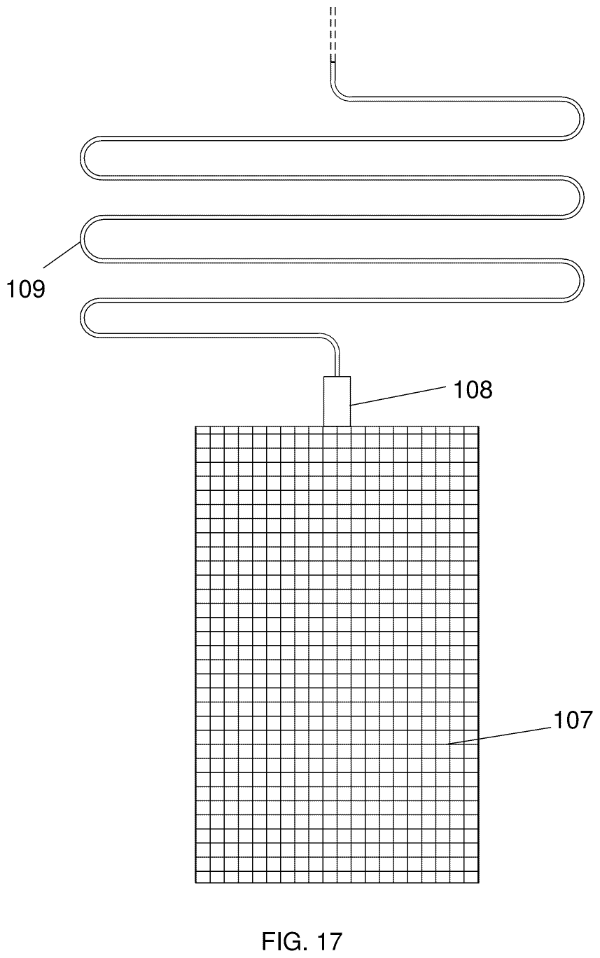

With reference to attached FIGS. 1-21 and specifically FIGS. 1-3, the wound closure patch (100) used on a subject (S), comprises an outer contractible layer (101), wherein an aperture (106) can be placed for access to the wound (W). The aperture (106) may be created via using a cutting tool or may be integral to the wound closure patch (100) examples of which are explained herein. The wound closure patch (100) may further comprise a pad (107) of flexible material, being in some examples a bandage or wound dressing material such as gauze, TELFAR, antimicrobial-infused material, a gauze dressing, a transparent film dressing, or a hydrocolloid dressing. As illustrated in FIGS. 1 and 17, the pad (107) may further comprise an anchor point (108) to which a tether (109) may be connected. The anchor point (108) may be an attachment point such as via gluing or tying, stapling, or in some other manner of affixing the tether (109) to the pad (107). Alternately the anchor point (108) may be a loop to which the tether (109) may be knotted, looped through, or otherwise connected to. The tether (109) may serve to move or direct the pad (107) into a central channel (110) formed in the wound closure patch (100).

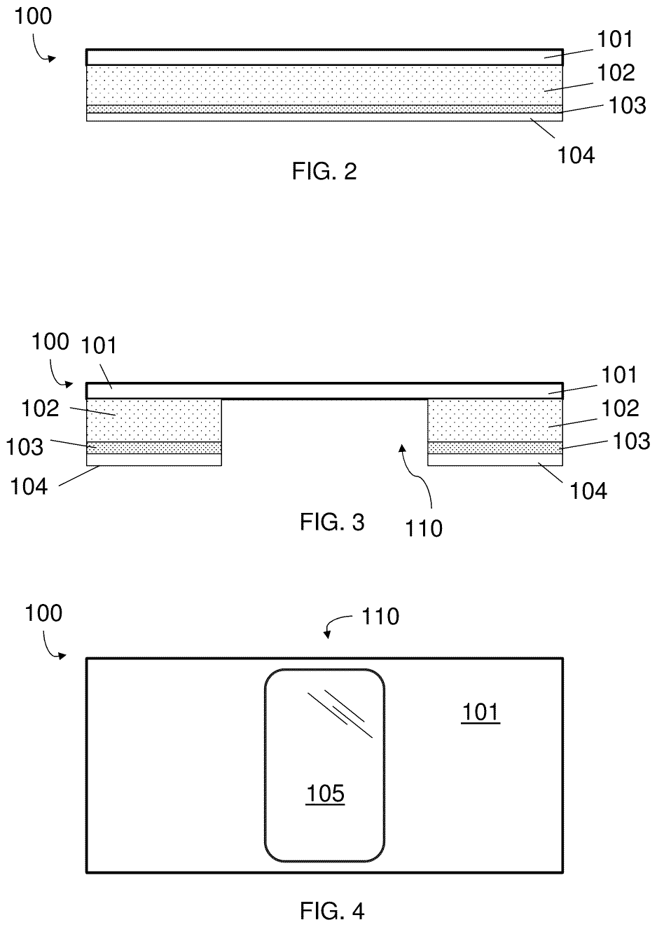

As illustrated in FIG. 2, which is illustrative of the layers of the wound closure patch (100), the wound closure patch (100) comprises the outer contractible layer (101), an intermediate layer (102) joined to the outer contractible layer (101), and an adhesive layer (103) joined to the intermediate layer (102). The wound closure patch (100) may further comprise a peelable layer (104), such as peelable paper or plastic, or film layer, which protects the adhesive layer (103).

The outer contractible layer (101) comprises a stimuli-responsive polymer, or otherwise termed a shrinkable polymer film or shape-memory polymer, which contracts when exposed to an activation stimulus. This activation stimulus may be specific to the stimuli-response polymer utilized, wherein the activation stimulus may include but is not limited to temperature, such as heat, via radiant, conductive, or convective heat, or pH, light, chemical, magnetic field, electrical field, or a humidity stimulus in non-limiting examples. The stimuli-responsive polymer may be selected from one or more of a polyethylene terephthalate (PET), polyolefin, shape-memory polymer, polylactic acid polymer, a polyurethane, and an ethylene-vinyl acetate polymer, or combinations thereof. In an example, the outer contractible layer (101) can comprise a heat-responsive polymer, wherein the polymer may be a thermal shrink polymer which comprises a plastic that shrinks when heat is applied. Thermal shrink polymers include but are not limited to a polyolefin, polyvinyl chloride (PVC), fluoropolymers including but not limited to polytetrafluoroethylene (PTFE) and fluorinated ethylene propylene (FEP), elastomers, being rubber-like polymers, and silicone or a fluoroelastomer (FKM).

In examples, the outer contractible layer (101) comprises one or more of a polyethylene terephthalate (PET), polyurethane film, a polyolefin film, a polyester film, a polynorbornene film, a polyvinyl acetate film, an epoxy-thermoset polymer film, a polymethyl methacrylate film, ethylene-vinyl acetate (EVA) a thiol-ene film, shape-memory polymers, and combinations thereof. The polyurethane film layer can be selected from the group consisting of: a thermoplastic polyurethane film, a polyester urethane film, a polyether urethane film, a polycarbonate urethane film, and combinations thereof. The polyolefin film may be selected from the group consisting of a polyethylene film, an ethylene-vinyl acetate film, a polyethylene-co-butylene film, and combinations thereof. Other additives such as a tinting, pigment, or coloring agent may be included. In other examples, the outer contractible layer (101) may be transparent or opaque.

When a thermal or heat stimulus is used to elicit the contraction response of the outer contractible layer (101), the thermal stimulus, which may include but is not limited to an infrared lamp, laser, controlled air heating tool, or chemical heat pack, which may comprise temperature sensors, visual indicators, and supply brief energy bursts of heat, may heat the outer contractible layer (101), being in one example a thermal shrink polymer, to a temperature of about 30 °C (about 86 °F), or about 40 °C, or about 50 °C, or about 60 °C, or about 70 °C, or about 80 °C (about 176 °F). In other examples, the thermal shrink polymer may be heated to about 30 °C, or about 35 °C, or about 40 °C, or about 45 °C, or about 50 °C, or about 55 °C, or about 60 °C, or about 65 °C, or about 70 °C, or about 75 °C, or about 80 °C. In other examples, temperature of the stimulus-responsive or thermal shrink polymer when stimulated may be about 35 °C to about 45 °C, about 40 °C to about 50 °C, about 45 °C to about 55 °C, about 50 °C to about 60 °C, about 55 °C to about 65 °C, about 60 °C to about 70 °C, and about 65 °C to about 75 °C.

The outer layer (101) may be contractible or responsive to other stimuli such as but not limited to a light stimulus, such as UV irradiation, a chemical stimulus, an electric or electric field stimulus, a magnetic field stimulus, and a humidity stimulus. When the outer contractible layer (101) comprises a UV or visible light stimulated material or polymer, the contractible layer (101) may be stimulated to contract via UV or visible light exposure. These polymers may comprise chromophores, in a non-limiting example azopolymers which incorporate azobenzenes, or spiro pyrans. Other light responsive polymers may contain cinnamoyl groups that form networks upon exposure to light. Other examples comprise metal complexes or metallopolymers, like ruthenium, that respond to light, changing shape upon light exposure or contracting to pull wound sides together.

A pH responsive material or polymer of the contractible layer (101) may comprise a polyacid or anionic containing acidic group like carboxylic (—COOH), sulfonic (—SO₃H), or phosphoric (—PO₄) acids. At low pH, below their pKa, these groups protonate, becoming positively charged causing the polymer to shrink. Examples of polyacid polymers include but are not limited to polyacrylic acid (PAA), poly methacrylic acid (PMAA), alginic acid, and hyaluronic acid. A pH responsive polymer may comprise a polybase or cationic containing group, such as but not limited to primary, secondary, or tertiary amines (—NH₂), or aromatic nitrogen groups like pyridine and imidazole. These groups lose protons at high pH, above their pKa, and shrink. Examples of polybase polymers may include but are not limited to chitosan, polyethylene imine (PEI), poly(N,N-dimethylaminoethyl methacrylate) (PDMAEMA), and poly(4-vinylpyridine).

A chemical-responsive material or polymer of the contractible layer (101) may comprise materials that change physical or chemical properties like shape, solubility, and stiffness when exposed to chemical triggers such as pH, ions, glucose or other sugars, enzymes, specific ligands, and/or gases. Specific groups on a polymer chain might bind or react to specific molecules for instance boronic acid groups may bind glucose, or amine groups may bind CO₂ causing physical or other changes in the polymer.

Magnetic field-responsive polymers (MERPs), being examples of materials of the contractible layer (101), may comprise composite materials that may integrate magnetic nanoparticles like iron oxide into polymer matrices and change shape upon exposure to a magnetic field. Magnetic nanoparticle examples may comprise iron oxide (Fe₃O₄), or cobalt ferrite (CoFe₂O₄) dispersed within for example chitosan, polydimethylsiloxane (PDMS), or PLA.

Electrical field-responsive polymers (ERPs), being examples of materials of the contractible layer (101), may comprise materials that can change shape, volume, or other properties when exposed to an electric field. Ionic ERP materials contain charged groups, in non-limiting examples sulfates or carboxylates, that move or re-orient upon exposure to an electrical field causing shrinking or bending under low voltage.

A humidity-responsive polymer (HR-polymer), being examples of materials of the contractible layer (101), may comprise materials that may change shape or size when exposed to moisture or conversely stripped of moisture, i.e. dried or dehydrated. Hydrophilic groups like carboxyls in PVA or PAA hydrogels, or starch, absorb or release water causing the polymer network to swell or shrink respectively, and therefore change dimensions. Examples of humidity-responsive polymers may include but are not limited to hydrogel blends such as PVAs and PAAs, starch-based polymers, liquid crystalline polymers (LCPs), and copolymers like polyethylene-co-acrylic acid (EAA).

The outer contractible polymer layer (101) may have a thickness of, for example, at least 0.10 mm, or about 0.10 mm, or about 0.20 mm to about 1.0 mm. In certain embodiments, the outer contractible stimuli-responsive polymer layer (101) may have a thickness of about 0.10 mm to 0.20 mm, about 0.15 mm to 0.25 mm, 0.20 mm to about 0.30 mm, about 0.25 mm to about 0.35 mm, about 0.30 mm to about 0.40 mm, about 0.35 mm to about 0.45 mm, about 0.40 mm to about 0.50 mm, about 0.45 mm to about 0.55 mm, about 0.50 mm to about 0.60 mm, about 0.55 mm to about 0.65 mm, about 0.60 mm to about 0.70 mm, about 0.65 mm to about 0.75 mm, about 0.70 mm to about 0.80 mm, about 0.75 mm to about 0.85 mm, about 0.80 mm to about 0.90 mm, and about 0.85 mm to about 0.95 mm.

With reference to FIG. 1, the outer contractible layer (101) may be designed to have a stimuli-responsive polymer composition so that when the stimuli is applied, wound edges of a wound (W) lying underneath the wound closure patch (100), preferably lying in the area of the central channel (110), are pulled together by the movement of the outer contractible layer (101) contracting. When the wound (W) edges are pulled together and held together by the outer contracting layer (101), wound closure and healing may be achieved in quicker time. As illustrated in FIG. 3, a central channel (110) is designed in a central portion of the wound patch (100). The outer contractible layer (101) covers the central channel (110) while the other two layers, the intermediate layer (102) and adhesive layer (103), flank the central channel (110). The central channel (110) may be of various shapes to accommodate a pad (107) and insertion of the pad (107) therein, including but not limited to wider outer edges. A peelable layer (104) may be joined, affixed, or attached to the adhesive layer (103). In the case of the embodiment illustrated in FIG. 2, two separate peelable layer portions (104), may be attached to the adhesive layer(s) (103), and wherein the peelable layer (104) may be peelable from the adhesive layer (103). Following placement of the wound closure patch (100) and contracting the outer contractible layer (101), the pad (107) may be fit into the central channel (110) to protect the wound. An aperture (106) or slit may be made in the outer contractible layer (101) to view the wound for instance after some period of time.

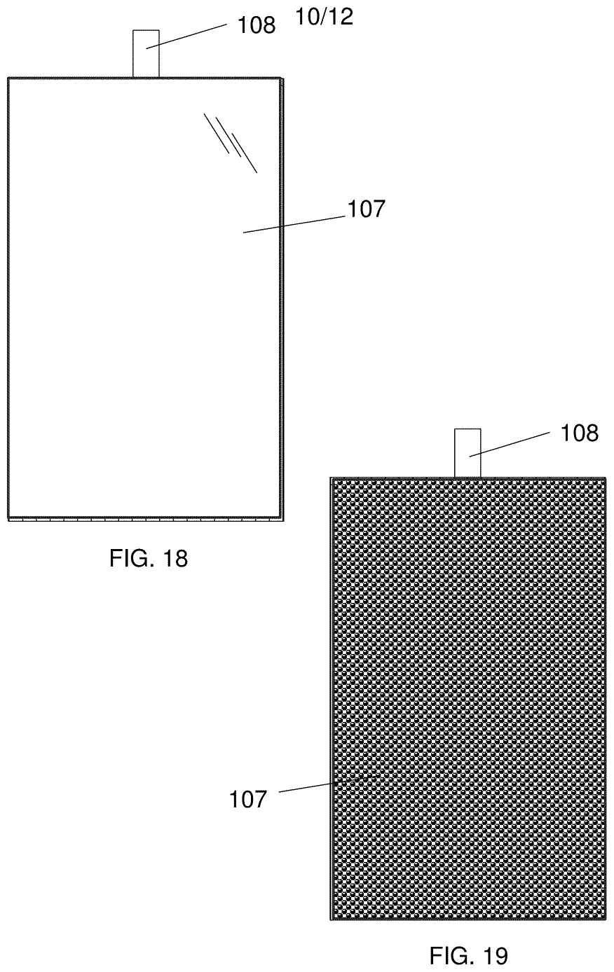

As illustrated in FIG. 1 and FIGS. 17-19, the pad (107) may comprise a wound (W) dressing, in an example being a gauze dressing, exampled in FIG. 17, a transparent film dressing, exampled in FIG. 18, or a hydrocolloid dressing, exampled in FIG. 19. The pad (107) may further comprise a medicament applied to the pad (107) wherein the medicament may be in non-limiting examples an antibiotic, antiseptic, such as polyhexamethylene biguanide (PHMB) POLYSPORIN® or NEOSPORIN® of Kenvue Brands LLC, an ointment, silver, honey, such as Manuka honey, aloe vera, curcumin, or a nonsteroidal anti-inflammatory (NSAID).

As illustrated in FIGS. 2-3, the intermediate layer (102) joined to the outer contractible layer (101) may comprise a thermal insulator, flexible foam, or hydrogel layer which may insulate the subject skin (SS) under the wound closure patch (100). The intermediate layer (102) and outer contractible layer (101) may be joined by methods known in the field such as via adhesive bonding, lamination, co-extrusion, cross-linking, and ultra-sonic assembly. The thermal insulator of the intermediate layer (102) may comprise an open cell foam, a closed cell foam, or a hydrogel. The intermediate layer (102) may comprise one or more of a polyurethane foam, a reticulated polyurethane (PU) foam, a hydrocellular foam, a silicone foam, a hydrofiber foam, alginate foam, a chitosan foam, a collagen foam, a hydrogel foam, a polyvinylpyrrolidone foam, a polyethylene glycol foam, a polylactic-co-glycolic acid foam, a polylactic acid foam, a polybutylene succinate foam, a polyhydroxyalkanoates foam, a poly-ε-caprolactone foam, a polyethylene foam, a cross-linked polyethylene foam, a polypropylene foam, a polyethylene-vinyl acetate foam, a polyvinyl chloride foam, a polyvinyl formal foam, a polyvinyl alcohol foam, a polyimide foam, a polystyrene foam, a melamine foam, a nitrile rubber foam, and a hydroxyapatite-based foams. The intermediate layer (102) may have a thickness of at least about 0.8 mm to about 1.0 mm to about 5.00 mm, or about 1.50 mm to about 2.50 mm, about 2.00 mm to about 3.00 mm, about 2.50 mm to about 3.50 mm, about 3.0 mm to about 4.0 mm, and about 3.5 mm to about 4.50 mm.

As illustrated in FIGS. 2-3, the adhesive layer (103), being in one example a skin-safe and/or medical adhesive layer, joined to the intermediate layer (102) may comprise a biocompatible adhesive layer which may comprise one or more of the following including but not limited to an acrylic-silicone hybrid adhesive, cyanoacrylate adhesive, a 2-octyl-cyanoacrylate adhesive, a n-butyl cyanoacrylate adhesive, a silicone adhesive, an acrylic adhesive, a UV-curable acrylate adhesive, a polyurethane adhesive, a hydrogel adhesive, a collagen adhesive, a chitosan adhesive, an albumin adhesive, a dextran adhesive, a gelatin adhesive, a poly ethylene glycol adhesive, a polyacrylic acid adhesive, and a polylactic-co-glycolic adhesive. The adhesive layer (103) would be joined or applied to the intermediate layer (102) during manufacture via standard manufacturing procedures appropriate for intermediate layer (102) and adhesive layer (103) composition and may include applying via brushing, spraying, or otherwise applying an adhesive film. The adhesive layer (103) may be less than about 0.1 mm thick, or less than about 0.2 mm thick, or less than about 0.3 mm thick, or less than about 0.4 mm thick, or less than about 0.5 mm thick, or less than about 0.6 mm thick, or about 0.7 mm thick. The adhesive may be breathable or water-resistant.

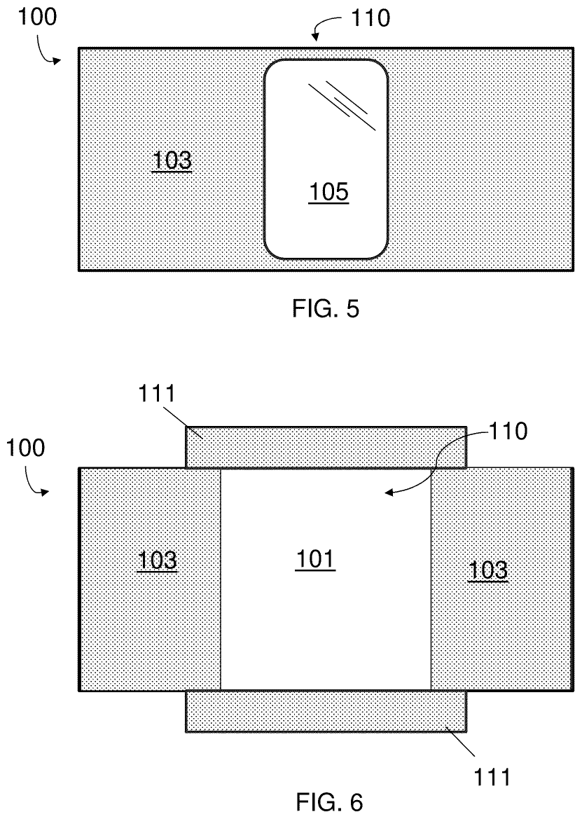

As illustrated in FIGS. 4-5, an embodiment of the wound closure patch (100) may comprise a transparent window (105) in the contractible outer layer (101) from which to view the wound (W). The transparent window (105) may be positioned by a user or medical aid over a wound (W). The transparent window (105) may necessarily lie above the central channel (110) that may also be positioned over a wound (W) flanked by or partially flanked by intermediate layer (102) and adhesive layer (103). The transparent window (105) may be comprised of a transparent polymer material such as but not limited to an acrylic (PMMA), polycarbonate, polystyrene, polypropylene, and PVC. In an embodiment, the aperture FIG. 1, (106) may be made in the transparent window. In other embodiments, the outer contractible layer (101) may be manufactured with an aperture (106) comprised therein and wherein an additional outer contractible layer or a transparent window may be applied over and joined at edges of the additional outer contractible layer with integral adhesive joined or applied during manufacturing.

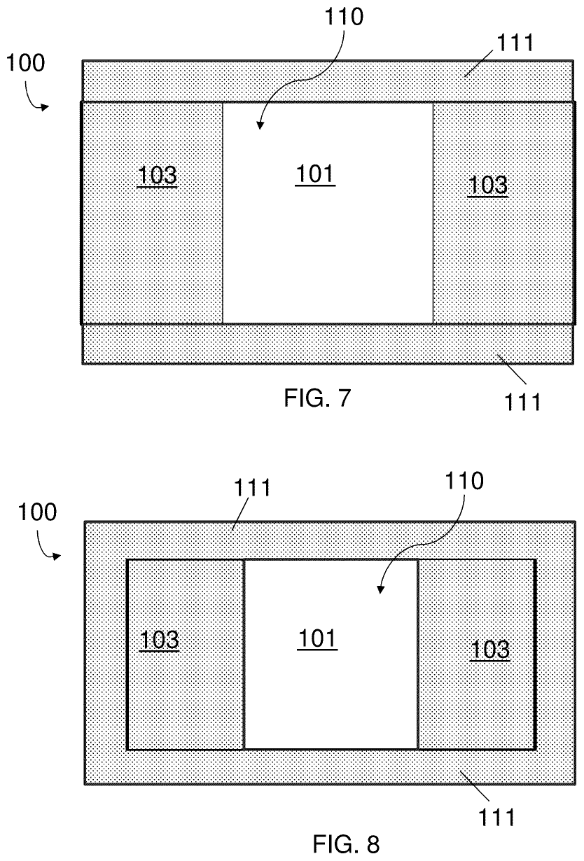

As illustrated in FIGS. 6-8, the wound closure patch (100) may in embodiments comprise at least one additional wing (111), and in some embodiments at least two or more additional wings (111). The wing(s) (111) may at least span the central channel (110) as shown in FIG. 6 such that air flow into the central channel (110) and wound area is limited. The adhesive backed wing(s) (111) may comprise a layer and an adhesive applied to a back or underside portion of the adhesive backed wing(s) (111) that may be designed to adjoin to the subject (S), being the subject's skin (SS). As illustrated in FIG. 7, the wing(s) (111) may span at least one entire side of the wound closure patch (100). As illustrated in FIG. 8, the wing(s) (111) may completely ring or surround the wound closure patch (100). A peelable layer may be applied to the adhesive of the adhesive backed wing(s) (111).

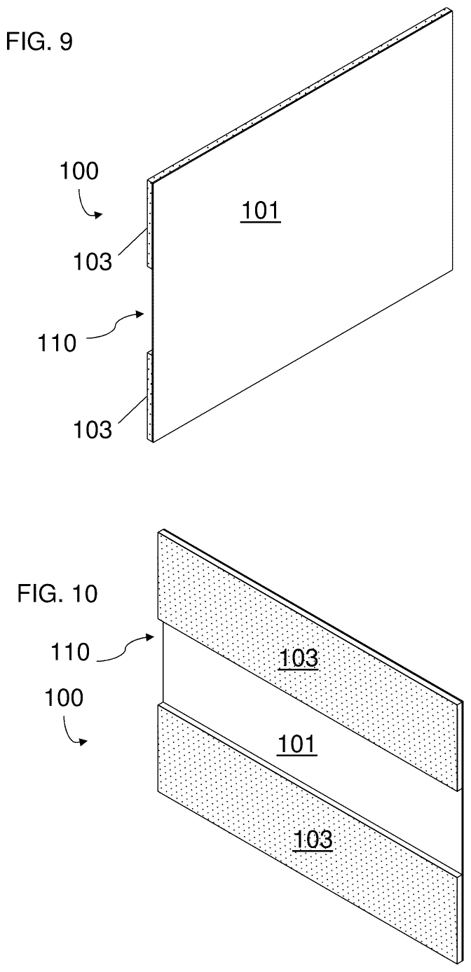

FIGS. 9-16 illustrate various views of the wound closure patch (100) of the present disclosure. The shape of the wound closure patch (100), which has been represented here as essentially square or rectangular, is non-limiting, i.e. is not limited to the shape presented herein, as the wound closure patch (100) may be designed in other shapes and sizes to accommodate varying size wounds (W), subject (S) wound area, and/or wound closure patch (100) placement options. Shapes envisioned for the wound closure patch (100) may be, in non-limiting examples, symmetrical, being a circle, oval, triangle, square, rectangle, diamond, or, in other examples, an asymmetrical shape. The wound closure patch (100) may be designed to be shaped by the user or medical aid applying the wound closure patch (100) such as via cutting to a certain size to accommodate placement and size of wound (W). The wound patch (100) may be pre-sterilized and packaged for medical aid use or consumer use. The wound patch (100) may be manufactured in various sizes to be used with various sized wounds (W) and/or subjects (S).

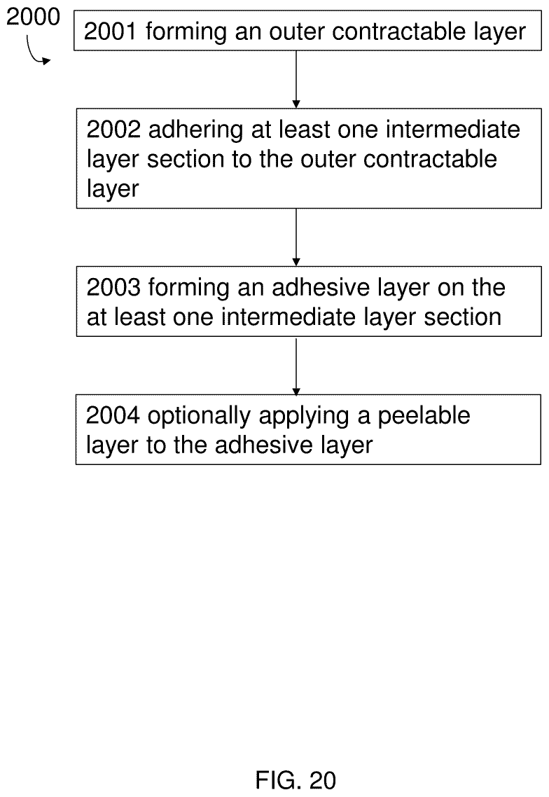

Now, with primary reference to FIG. 20, the present disclosure encompasses a method 2000 for making a wound closure patch. The method 2000 comprises as least the steps of: a) 2001 forming an outer contractible layer (101); b) 2002 adhering at least one intermediate layer section (102) to the outer contractible layer (101); and c) 2003 forming or joining an adhesive layer (103) to or on the at least one intermediate layer section (102). The method may further optionally comprise 2004 applying a peelable layer (104) to the adhesive layer (103). The method may optionally further comprise applying adhesive backed wings (111) to the wound closure patch (100), wherein the adhesive backed wings (111) are formed on or applied to the outer contractible layer (101). The method may further comprise making a transparent window (105) in the outer contractible layer (101). The method may further comprise wherein a peelable layer (104) may be applied to the adhesive back of the adhesive backed wings (111). Compositions of layers and materials for manufacture are described above, as well as general manufacturing methods which would necessarily be designed with materials of manufacture in mind. The wound closure patch (100) may be sterilized. The wound closure patch (100) may be sterilized and packaged in sterilized packaging for use or sale.

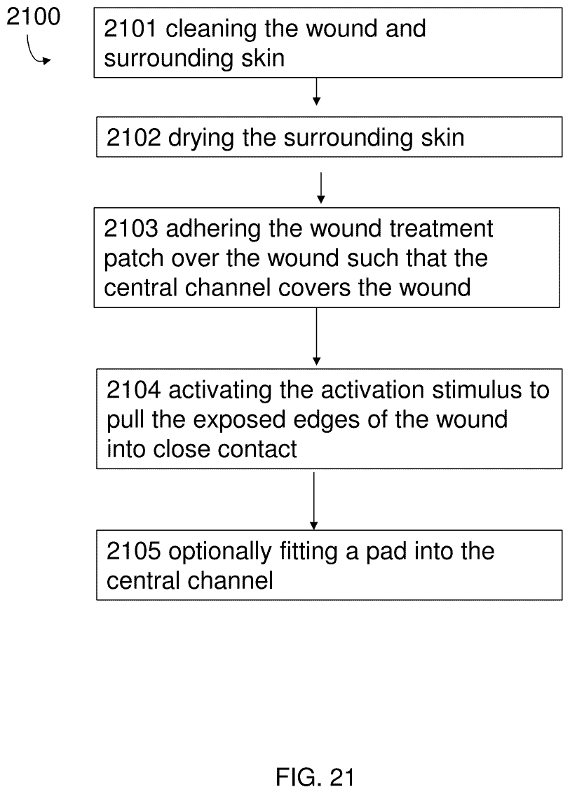

Now, with primary reference to FIG. 21, the present disclosure encompasses a method (2100) for using a wound closure patch (100) of the present disclosure. The method (2100) comprises a method for treating a wound (W) with exposed edges comprising cleaning the wound (W) and surrounding subject's skin (SS) (2101), optionally approximating the size of the wound (W) and choosing size of wound patch (100) to be applied, drying the surrounding skin (2102), adhering the wound treatment patch (100) over the wound such that the central channel covers the wound (2103), optionally applying benzoin, Mastisol® of Ferndale Laboratories, or a skin adhesive promoter, and activating the activation stimulus (2104) to contract the outer contractible layer (101) of the wound closure patch (100) thereby pulling the exposed edges of the wound (W) into close contact. A central window may be trimmed to the size of the wound (W) or an aperture (106) may be formed in the contractible outer layer (101). Further, and optionally, a pad (107), with or without medicament, may be fit 2105 into the central channel (110) of the wound closure patch (100). The pad (107) may be replaced at regular intervals. The patch (100) may be left in place for at least a few hours, at least six hours, at least nine hours, or at least twelve hours, or at least one day to at least two days, to at least three days, being several days for healing. The wound (W) may be cleaned at regular intervals. Additional methods of use may comprise but are not limited to creating an aperture (106) in the outer contractible layer (101). A used wound closure patch (100) may be removed and a new wound closure patch (100), or new pad (107) may be placed at regular intervals.

As may be easily understood from the foregoing, the basic concepts of the present invention may be embodied in a variety of ways. The invention involves numerous and varied embodiments of a wound closure patch and methods for making and using such a wound closure patch.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms may be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a wound closure patch should be understood to encompass disclosure of the act of making or using the wound closure—whether explicitly discussed or not—and, conversely, were there effectively disclosure of the act of making or using the wound closure patch, such a disclosure should be understood to encompass disclosure of a wound closure patch and even a means for treating a wound. Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” may be used interchangeably herein.

Further, for the purposes of the present invention, the term “coupled” or derivatives thereof may mean indirectly coupled, coupled, directly coupled, connected, directly connected, or integrated with, depending upon the embodiment.

Additionally, for the purposes of the present invention, the term “integrated” or “joined” when referring to two or more components means that the components (i) may be united to provide a one-piece construct, a monolithic construct, or a unified whole, or (ii) may be formed as a one-piece construct, a monolithic construct, or a unified whole. Said another way, the components may be integrally formed, meaning connected together so as to make up a single complete piece or unit, or so as to work together as a single complete piece or unit, and so as to be incapable of being easily dismantled without destroying the integrity of the piece or unit.

Thus, the applicant(s) should be understood to claim at least: i) each of the embodiments of a wound closure patch herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The elements following an open transitional phrase such as “comprising” may in the alternative be claimed with a closed transitional phrase such as “consisting essentially of” or “consisting of” whether or not explicitly indicated the description portion of the specification.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A wound closure patch, comprising:
an outer contractible layer extending to a contractible layer periphery;
an intermediate layer joined to said outer contractible layer, said intermediate layer including a pair of segments disposed in spaced apart relation defining a central channel having opposite channel ends open at said contractible layer periphery; and
an adhesive layer joined to said intermediate layer.

2. The wound closure patch of claim 1, further comprising an aperture open between opposed surfaces of said outer contractible layer, said aperture disposed in said outer contractible layer over said central channel.

3. The wound closure patch of claim 1, wherein said outer contractible layer comprises a stimuli-responsive polymer which contracts when exposed to an activation stimulus.

4. (canceled)

5. (canceled)

6. The wound closure patch of claim 1, wherein said outer contractible layer comprises a thermal shrink polymer which contracts in response to a thermal stimulus.

7. (canceled)

8. The wound closure patch of claim 6, wherein said thermal stimulus heats said thermal shrink polymer to a temperature of about 30 °C (about 86 °F) to about 80 °C (about 176 °F).

9. (canceled)

10. The wound closure patch of claim 1, wherein said outer contractible layer has a thickness of about 0.20 mm to about 1.0 mm.

11-14. (canceled)

15. The wound closure patch of claim 1, wherein said intermediate layer joined to said outer contractible layer comprises a thermal insulator.

16. The wound closure patch of claim 15, wherein said thermal insulator comprises one or more of an open cell foam, a closed cell foam, a hydrogel, and combinations thereof.

17. (canceled)

18. The wound closure patch of claim 1, wherein said intermediate layer has a thickness of about 1.00 mm to about 5.00 mm.

19. (canceled)

20. The wound closure patch of claim 1, wherein said adhesive layer comprises a biocompatible adhesive layer.

21. (canceled)

22. The wound closure patch of claim 1, further comprising a pad disposed in said central channel.

23. The wound closure patch of claim 22, wherein said pad comprises a wound dressing.

24. The wound closure patch of claim 23, wherein said wound dressing comprises one or more of: a gauze dressing, a transparent film dressing, a hydrocolloid dressing.

25. The wound closure patch of claim 22, further comprising a tether connected to said pad, said tether configured to pass through said central channel, said pad drawn by said tether into said central channel.

26. The wound closure patch of claim 22, further comprising a medicament disposed on said pad.

27. The wound closure patch of claim 26, wherein said medicament comprises one or more of: an antibiotic, an antiseptic, an ointment, silver, honey, aloe vera, curcumin, and nonsteroidal anti-inflammatory.

28. The wound closure patch of claim 1, further comprising a peelable layer joined to said adhesive layer, said peelable layer peelable from said adhesive layer.

29. The wound closure patch of claim 1, further comprising adhesive backed wings attached to the contractible layer periphery and flanking each of the opposite channel ends.

30. The wound closure patch of claim 29, wherein the adhesive backed wings comprise a peelable layer attached to the adhesive.

31. The wound closure patch of claim 1, wherein the outer contractible layer comprises a transparent portion covering at least a portion of the central channel.

32-41. (canceled)
